(19) United States
(12) Patent Application Publication
(10) Pub. No.: US 2025/0194940 A1
(43) Pub. Date: Jun. 19, 2025

(54) DEVICE AND METHOD FOR ESTIMATING BLOOD PRESSURE IN NON-CONTACT MANNER

(71) Applicant: Electronics and Telecommunications Research Institute, Daejeon (KR)

(72) Inventors: Tae Wuk BAE, Daejeon (KR); Kee Koo KWON, Daejeon (KR); Eun Chang CHOI, Daejeon (KR)

(73) Assignee: Electronics and Telecommunications Research Institute, Daejeon (KR)

(21) Appl. No.: 18/987,255

(22) Filed: Dec. 19, 2024

(30) Foreign Application Priority Data
Dec. 19, 2023 (KR) 10-2023-0185960

(51) Int. Cl. A61B 5/021; A61B 5/00; A61B 5/024

(52) U.S. Cl. CPC A61B 5/021; A61B 5/02405; A61B 5/02416; A61B 5/7267; A61B 5/7475

(57) ABSTRACT

Provided are a device and method for estimating blood pressure in a non-contact manner. The device includes a memory, an imaging module that captures a body part image of a user, and a processor connected to the memory and the imaging module. The processor extracts a remote photoplethysmography (rPPG) signal from the body part image, detects a plurality of peak values from the rPPG signal, extracts heart rate variability (HRV) parameters using the detected peak values, and estimates blood pressure of the user on the basis of the HRV parameters.

### CROSS-REFERENCE TO RELATED APPLICATION

This application claims priority to and the benefit of Korean Patent Application No. 10-2023-0185960, filed on Dec. 19, 2023, the disclosure of which is incorporated herein by reference in its entirety.

### BACKGROUND

### 1. Field of the Invention

The present invention relates to a device and method for estimating blood pressure in a non-contact manner using remote photoplethysmography (rPPG).

### 2. Discussion of Related Art

The most accurate method of measuring blood pressure may be inserting a needle in a blood vessel for measurement. However, this method may be a significant burden for patients and may not be an easy way of measurement.

Currently, blood pressure measurement may be done in a non-invasive, needle-free way using a sphygmomanometer. In other words, a sphygmomanometer is used to measure diastolic and systolic blood pressure.

However, the contact blood pressure measurement method according to the related art requires a break of at least 15 minutes between measurements, which makes it difficult to measure blood pressure consecutively and continuously.

In addition, it is necessary to wear a sphygmomanometer, which is relatively voluminous and difficult to carry, and it is necessary to wear a cuff every time a user measures his or her blood pressure, which is cumbersome.

### SUMMARY OF THE INVENTION

The present invention is directed to providing a device and method for estimating blood pressure in a non-contact manner that allow non-contact blood pressure measurement by extracting remote photoplethysmography (rPPG) from an image captured through an imaging module (e.g., a camera).

According to an aspect of the present invention, there is provided a device for estimating blood pressure in a non-contact manner, which includes a memory, an imaging module configured to capture a body part image of a user, and a processor connected to the memory and the imaging module. The processor extracts an rPPG signal from the body part image, detects a plurality of peak values from the rPPG signal, extracts heart rate variability (HRV) parameters using the detected peak values, and estimates blood pressure of the user on the basis of the HRV parameters.

The body part image may include a facial image of the user.

The processor may extract HRV parameters of a time domain and HRV parameters of a frequency domain using the peak values of the rPPG signal.

The processor may input the HRV parameters to a deep learning model to classify the blood pressure of the user as at least one of low blood pressure, normal blood pressure, and high blood pressure.

The processor may input value of the HRV parameters of a specific time or log values of the HRV parameters of the specific time to the deep learning model.

The processor may input time-series variation data of the HRV parameters to a time-series deep learning model to estimate diastolic blood pressure and systolic blood pressure.

The time-series deep learning model may be a model generated through transfer learning of a pretrained PPG blood pressure estimation model.

The time-series deep learning model may use a pretrained feature extractor of the pretrained PPG blood pressure estimation model and may be a model generated by newly training a classifier using rPPG signals.

The time-series deep learning model may use a part of a pretrained feature extractor of the pretrained PPG blood pressure estimation model and may be a model generated by newly training a part of the feature extractor and a classifier using rPPG signals.

The time-series deep learning model may be a model generated by newly training a feature extractor and a classifier using rPPG signals.

The device may further include a user interface unit, and the processor may output information about the estimated blood pressure through the user interface unit.

According to another aspect of the present invention, there is provided a method of estimating blood pressure in a non-contact manner, which includes extracting, by a processor, an rPPG signal from a body part image of a user acquired through an imaging module, detecting, by the processor, a plurality of peak values from the rPPG signal, extracting, by the processor, HRV parameters using the detected peak values, and estimating, by the processor, blood pressure of the user on the basis of the HRV parameters.

The body part image may include a facial image of the user.

The extracting of the HRV parameters may include extracting, by the processor, HRV parameters of a time domain and HRV parameters of a frequency domain using peak values of the rPPG signal.

The estimating of the blood pressure of the user may include inputting, by the processor, the HRV parameters to a deep learning model to classify the blood pressure of the user as at least one of low blood pressure, normal blood pressure, and high blood pressure.

The estimating of the blood pressure of the user may include inputting, by the processor, values of the HRV parameters of a specific time or log values of the HRV parameters of the specific time to the deep learning model.

The estimating of the blood pressure of the user may include inputting, by the processor, time-series variation data of the HRV parameters to a time-series deep learning model to estimate diastolic blood pressure and systolic blood pressure.

The time-series deep learning model may be a model generated through transfer learning of a pretrained PPG blood pressure estimation model.

The time-series deep learning model may use a pretrained feature extractor of the pretrained PPG blood pressure estimation model and may be a model generated by newly training a classifier using rPPG signals.

The time-series deep learning model may use a part of a pretrained feature extractor of the pretrained PPG blood pressure estimation model and may be a model generated by newly training a part of the feature extractor and a classifier using rPPG signals.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a schematic block diagram of a device for estimating blood pressure in a non-contact manner according to an exemplary embodiment of the present invention;

FIG. 2 is a graph illustrating peak detection from a remote photoplethysmography (rPPG) signal according to an exemplary embodiment of the present invention;

FIG. 3 is a set of graphs illustrating heart rate variability (HRV) according to an exemplary embodiment of the present invention;

FIG. 4 is a table illustrating HRV parameters according to an exemplary embodiment of the present invention;

FIG. 5 is a diagram illustrating blood pressure classification based on HRV parameters according to an exemplary embodiment of the present invention;

FIG. 6 is a diagram illustrating blood pressure estimation based on time-series variation data of HRV parameters according to an exemplary embodiment of the present invention;

FIGS. 7A and 7B are diagrams illustrating a pretrained PPG blood pressure estimation model and an rPPG blood pressure estimation model based on transfer learning according to an exemplary embodiment of the present invention; and

FIG. 8 is a flowchart illustrating a method of estimating blood pressure in a non-contact manner according to an exemplary embodiment of the present invention.

### DETAILED DESCRIPTION OF EXEMPLARY EMBODIMENTS

Hereinafter, a device and method for estimating blood pressure in a non-contact manner according to the present invention will be described with reference to the accompanying drawings. In this process, the thicknesses of lines, the sizes of components, and the like shown in the drawings may be exaggerated for the purpose of clarity and convenience of description. Also, terms to be described below are defined in consideration of functions in the present invention, and the terms may vary depending on the intention of a user or operator or precedents. Therefore, these terms are to be defined on the basis of the overall content of the specification.

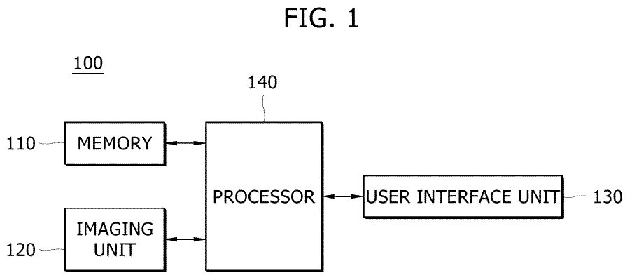

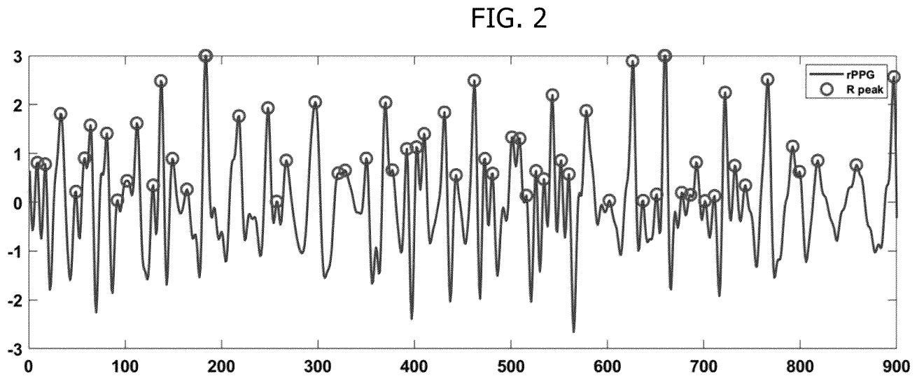

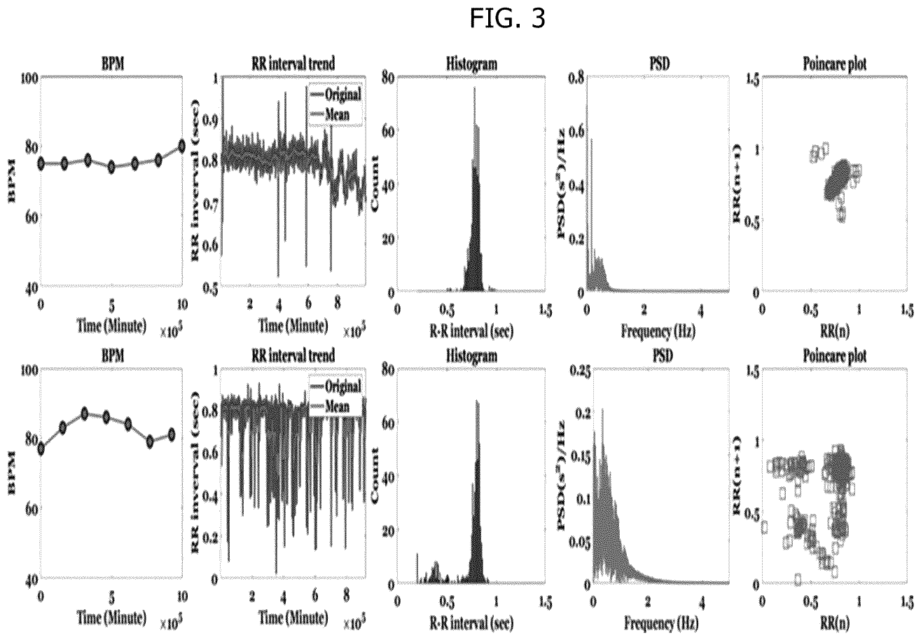

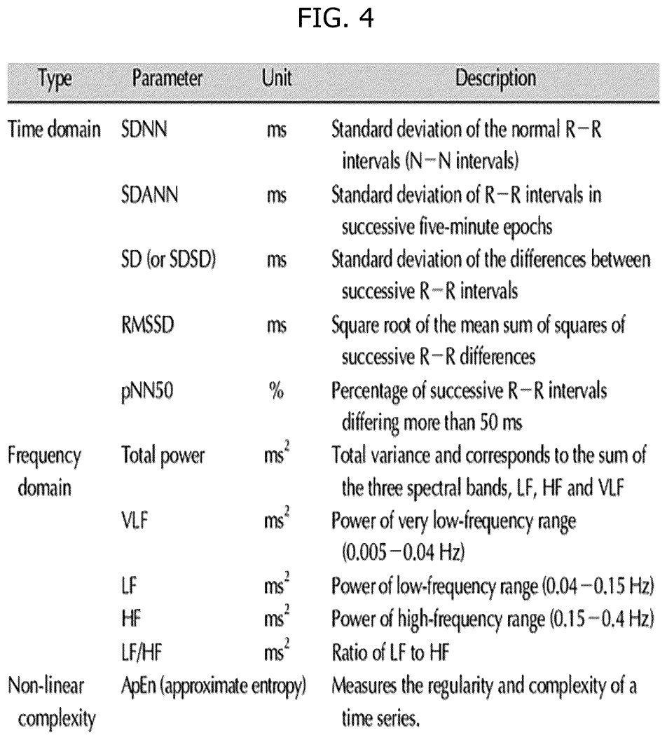

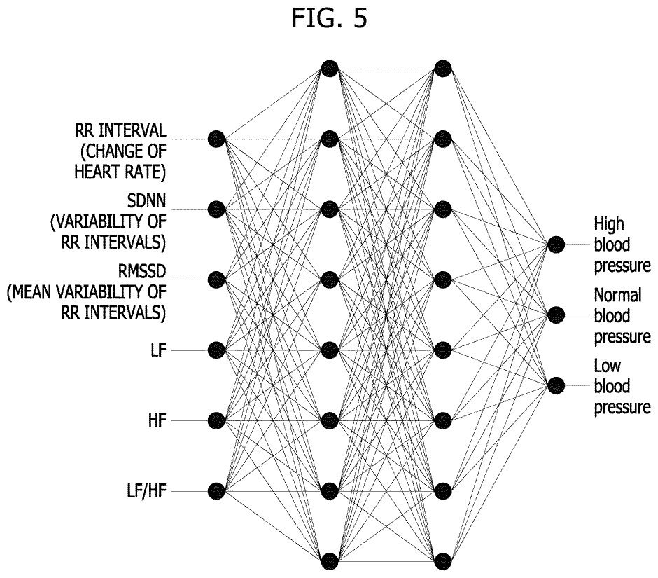

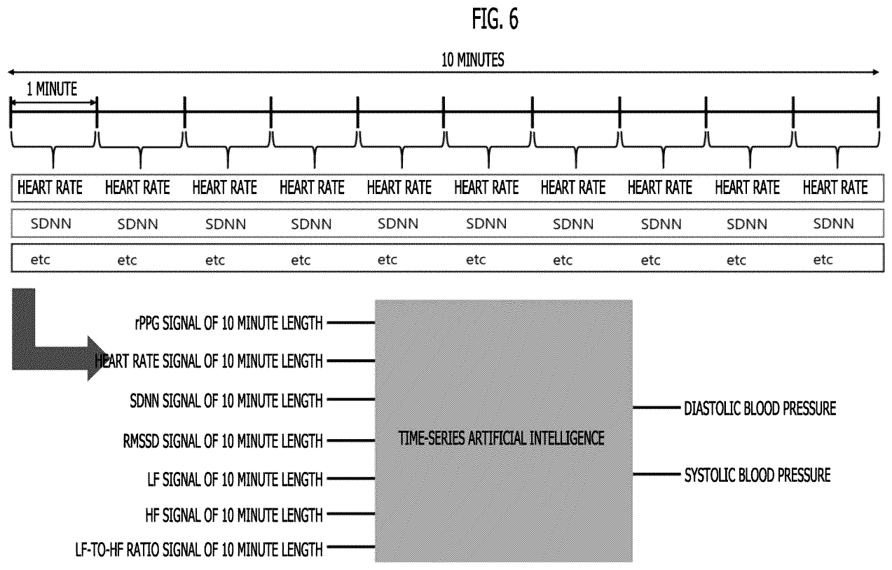

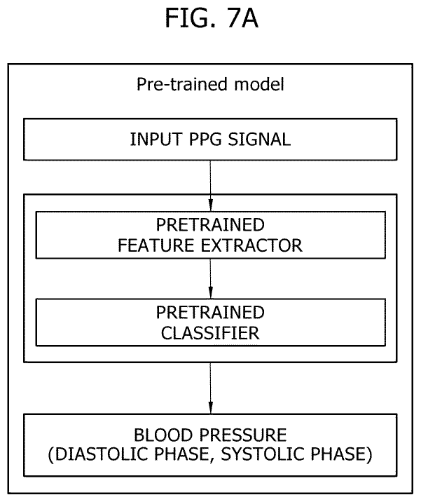

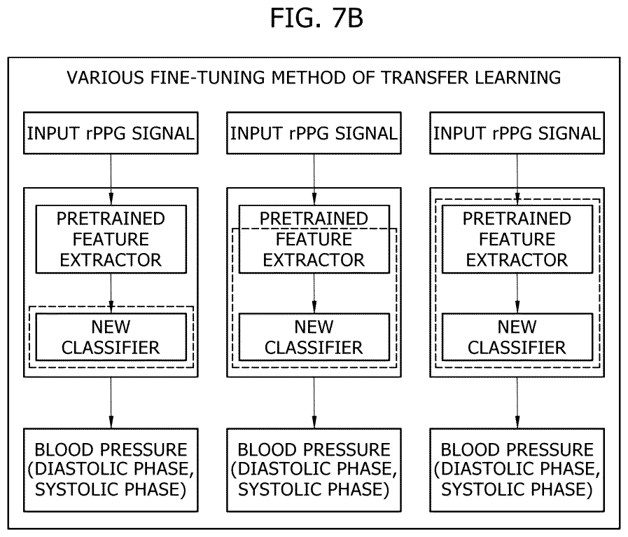

FIG. 1 is a schematic block diagram of a device for estimating blood pressure in a non-contact manner according to an exemplary embodiment of the present invention, FIG. 2 is a graph illustrating peak detection from a remote photoplethysmography (rPPG) signal according to an exemplary embodiment of the present invention, FIG. 3 is a set of graphs illustrating heart rate variability (HRV) according to an exemplary embodiment of the present invention, FIG. 4 is a table illustrating HRV parameters according to an exemplary embodiment of the present invention, FIG. 5 is a diagram illustrating blood pressure classification based on HRV parameters according to an exemplary embodiment of the present invention, FIG. 6 is a diagram illustrating blood pressure estimation based on time-series variation data of HRV parameters according to an exemplary embodiment of the present invention, and FIGS. 7A and 7B are diagrams illustrating a pretrained PPG blood pressure estimation model and an rPPG blood pressure estimation model based on transfer learning according to an exemplary embodiment of the present invention.

Referring to FIG. 1, a device 100 for estimating blood pressure in a non-contact manner according to the exemplary embodiment of the present invention may include a memory 110, an imaging unit 120, a user interface unit 130, and a processor 140.

The memory 110 is an element that stores data related to operations of the device 100 for estimating blood pressure in a non-contact manner. In particular, the memory 110 may store a program (application or applet) for extracting an rPPG signal from a facial image of a user, detecting a plurality of peak values from the rPPG signal, extracting HRV parameters using the detected peak values, and estimating blood pressure of the user on the basis of the HRV parameters, and the stored information may be selected by the processor 140 as necessary. Also, the memory 110 may store a deep learning model for classifying blood pressure as at least one of low blood pressure, normal blood pressure, and high blood pressure and a time-series deep learning model for estimating diastolic blood pressure and systolic blood pressure using time-series variation data of HRV parameters. In addition, the memory 110 may store an operating system (OS) for operating the device 100 for estimating blood pressure in a non-contact manner. Here, the memory 110 collectively refers to non-volatile storage devices that continuously maintain stored information without power supply and volatile storage devices that require power to maintain stored information. The memory 110 may perform a function of temporarily or permanently storing data processed by the processor 140. In addition to the volatile storage devices, the memory 110 may include magnetic storage media or flash storage media, but the present invention is not limited thereto.

The imaging unit 120 may capture a body part image of the user. Here, the body part image may be the facial image.

The imaging unit 120 may include a camera included in the device 100 for estimating blood pressure in a non-contact manner or an external general camera, infrared camera, zoom camera, etc.

The device 100 for estimating blood pressure in a non-contact manner may acquire an image that includes the user's facial skin and is captured using the camera included in the device 100 for estimating blood pressure in a non-contact manner or the external general camera, infrared camera, zoom camera, etc. The image including the user's face may be a video or consecutive photographs at regular time intervals in which the user's face is continuously shown at the same position. For example, when the device 100 for estimating blood pressure in a non-contact manner is installed in a smartphone, it is possible to acquire an image of the user's face using the smartphone.

The user interface unit 130 may provide a user interface for estimating blood pressure of the user.

For example, the user interface unit 130 may receive data or a control instruction required for the processor 140 to estimate blood pressure and may transmit the data or control instruction to the processor 140. The user interface unit 130 may also output a blood pressure classification result, a blood pressure estimation result, or the like of the processor 140.

The user interface unit 130 may be provided as a user interface such as a keyboard, a mouse, a touchpad, a touchscreen, an electronic pen, a touch button, etc. In addition, the user interface unit 130 may include a printer, a display, or the like to output data. Here, the display may be implemented as a thin film transistor-liquid crystal display (TFT-LCD) panel, a light-emitting diode (LED) panel, an organic LED (OLED) panel, an active matrix OLED (AMOLED) panel, a flexible panel, etc.

The processor 140 may control overall operations of the device 100 for estimating blood pressure in a non-contact manner. For example, the processor 140 may execute software (e.g., a program) stored in the memory 110 and control components (e.g., at least one of the memory 110, the user interface unit 130, and the imaging unit 140) connected to the processor 140. The processor 140 may be, but is not limited to, an application-specific integrated circuit (ASIC), a digital signal processor (DSP), programmable logic devices (PLDs), field programmable gate arrays (FPGAs), a central processing unit (CPU), microcontrollers, microprocessors, and/or the like.

The processor 140 may extract an rPPG signal from a body part image captured through the imaging unit 120, detect a plurality of peak values from the rPPG signal, extract HRV parameters using the detected peak values, and estimate blood pressure of the user on the basis of the HRV parameters. The body part image may be a facial image.

Operations of the processor 140 will be described in detail below.

When a facial image is received from the imaging unit 120, the processor 140 may perform preprocessing operations such as detecting a user's face from the facial image, detecting skin color, etc.

For example, the processor 140 may utilize a detection model, such as the Haar cascade, the histogram of oriented gradients (HOG), the single shot multibox detector (SSD), you only look once (YOLO) v3, or the like, to detect a facial region from the facial image, and may select and use an appropriate detection model of which setting values may be changed (limiting a detection region, changing input size, setting a threshold value, etc.) in accordance with an environment of the captured image.

When a facial region is detected, the processor 140 may extract an rPPG signal from the facial image. The rPPG signal may be an imaging PPG (iPSPG) or non-contact PPG (ncPPG) that allows non-contact monitoring of human cardiac activity through the imaging unit 120.

For example, the processor 140 may sample an image frame from facial images at specific time intervals, classify colors of pixels of the image frame, and calculate a ratio of a specific color in the image frame using the colors of the pixels, thus extracting an rPPG signal.

Also, the processor 140 may extract an rPPG signal after refining brightness variations of a skin region in the facial image.

To extract an rPPG signal from the facial image, the processor 140 may use various methods according to the related art.

An rPPG signal may be a similar signal to a PPG signal that is obtained from an oxygen saturation monitor through signal decomposition after an average color signal is extracted from each frame of captured facial images. rPPG signals have slightly unstable signal heights and cycles compared to general PPG signals. However, rPPG signals allow non-contact extraction of heart rate information.

When an rPPG signal is extracted, the processor 140 may detect a plurality of peak values from the rPPG signal.

For example, the processor 140 may detect R-peak values using a QRS wave detection algorithm. The processor 140 may detect a plurality of R waves during a measurement time using the QRS wave detection algorithm and sequentially detect R-peak values which are peak values of the R waves. Also, the processor 140 may detect R-peak values using the Pan & Tompkins algorithm or the Hamilton & Tompkins algorithm.

As shown in FIG. 2, the processor 140 may detect peak (R-peak) values from the rPPG signal.

When the peak values of the rPPG signal are detected, the processor 140 may extract HRV parameters using the detected peak values.

HRV represents the variation in between heartbeats, and a variation of heartbeat cycle may be detected by measuring the instantaneous cycles of heartbeats. HRV may reflect interaction between sympathetic nerves and parasympathetic nerves.

The processor 140 may generate an R-peak to R-peak interval (RRI) time-series signal RPn on the basis of the sequentially detected R-peak values. The processor 140 may calculate RRIs which are intervals sequentially detected between the R-peak values over time. The processor 140 may generate the RRI time-series signal RPn corresponding to an RRI tachogram by interpolating the RRIs. For example, the processor 140 may generate the RRI time-series signal RPn thorough interpolation for converting continuous RRI information into a sampling density distribution of RRI durations or a sampling density distribution of differences between adjacent RRIs.

Meanwhile, when R peaks are detected from an electrocardiogram (ECG) signal and used to extract HRV, HRV including beats per minute (BPM), RRIs (intervals between R peaks), an RR histogram, an RR power spectral density (PSD), a Poincare plot (a graph of a current RRI and a subsequent RRI), and the like may be extracted as shown in FIG. 3. In the present invention, peaks may be detected from an rPPG and used to generate HRV. Despite some difference between HRV generated from an ECG signal and HRV generated from an rPPG signal, the two kinds of HRV are assumed to have a similar characteristic overall.

The processor 140 may extract HRV parameters of the time domain and HRV parameters of the frequency domain using the peak values of the rPPG signal.

For example, the processor 140 may extract HRV parameters of the time domain and HRV parameters of the frequency domain as shown in FIG. 4.

The HRV parameters of the time domain may include standard deviation of normal N-N intervals (NNIs) (SDNN), standard deviation of NNIs in successive five-minute epochs (SDANN), standard deviation of differences between successive RRIs (SD or SDSD), square root of the mean sum of squares of successive RRIs (RMSSD), percentage of successive NNIs differing more than 50 ms (pNN50), etc. The HRV parameters of the frequency domain may include total power (TP), the power of very low-frequency range (VLF), the power of low-frequency range (LF), the power of high-frequency range (HF), an LF-to-HF (LF/HF) ratio, etc.

When HRV parameters are extracted, the processor 140 may estimate blood pressure of the user on the basis of the HRV parameters. In this case, the processor 140 may classify the blood pressure of the user as at least one of low blood pressure, normal blood pressure, and high blood pressure on the basis of HRV parameters of a specific time. Also, the processor 140 may estimate diastolic blood pressure and systolic blood pressure using time-series variation data of HRV parameters.

Specifically, the processor 140 may input the HRV parameters to a deep learning model to classify the blood pressure of the user as at least one of low blood pressure, normal blood pressure, and high blood pressure. In this case, the processor may input values of the HRV parameters of the specific time or log values of the HRV parameters of the specific time to the deep learning model to classify the blood pressure of the user as at least one of low blood pressure, normal blood pressure, and high blood pressure. The deep learning model may vary such as a multi-layer perceptron (MLP), a convolutional neural network (CNN), a generative adversarial network (GAN), a recurrent neural network (RNN), an autoencoder (AE), a neural ordinary differential equation (ODE), etc. The number of inputs and the number of outputs of deep learning are adjustable.

For example, as shown in FIG. 5, the processor 140 may input the HRV parameters to an MLP model to classify the three kinds of blood pressure (low blood pressure, normal blood pressure, and high blood pressure). Here, a change of the heart rate, an SDNN, an RMSSD, an LF, an HF, an LF-to-HF ratio, and the like are used as input to the MLP model. The HRV parameters input to the MLP model may be values that are calculated at a specific time (e.g., two minutes, five minutes, ten minutes, one hour, or the like) or values obtained by taking the natural logarithm (or logarithm) of the values, and the number of inputs may be adjusted.

In general, a decrease in RRI variability generally represents autonomic nervous system dysfunction. In other words, a decrease in RRI variability may be the cause of hypertension.

The processor 140 may estimate diastolic blood pressure and systolic blood pressure using the time-series variation data of HRV parameters. In this case, the processor 140 may input the time-series variation data of HRV parameters to the time-series deep learning model to estimate diastolic blood pressure and systolic blood pressure. The time-series variation data may be data having a certain number of HRV parameters corresponding to certain time units in chronological order. For example, in the case of an HRV parameter in length of one minute, a total of ten HRV parameters corresponding to ten minutes may be time-series variation data. The time-series deep learning model may include a long short-term memory (LSTM), a bidirectional LSTM, etc.

For example, the time-series deep learning model for estimating blood pressure (diastolic blood pressure and systolic blood pressure) on the basis of HRV parameters may be the same as shown in FIG. 5. Referring to FIG. 5, it is assumed that an rPPG signal has a length of ten minutes, and the 10-minute original rPPG signal, heart rates, SDNNs, RMSSDs, LFs, HFs, and LF-to-HF ratios, which are a plurality of (e.g., ten) pieces of time-series data, may be calculated for an rPPG signal in length of one minute and used as inputs to the time-series deep learning model (time-series artificial intelligence). The number of inputs and the number of outputs of the time-series deep learning model are adjustable.

Meanwhile, many deep learning models for estimating blood pressure using a PPG signal (PPG blood pressure estimation models) have been developed. According to the present invention, it is possible to generate an rPPG blood pressure estimation model for estimating blood pressure on the basis of an rPPG signal through transfer learning of a pretrained PPG blood pressure estimation model. The rPPG blood pressure estimation model may be the time-series deep learning model.

For example, according to the present invention, an rPPG blood pressure estimation model may be generated through transfer learning of the pretrained PPG blood pressure estimation model shown in FIG. 7A. The pretrained PPG blood pressure estimation model may include a pretrained feature extractor and a pretrained classifier. As shown in FIG. 7B, the rPPG blood pressure estimation model may be generated through transfer learning of the pretrained PPG blood pressure estimation model. For example, the rPPG blood pressure estimation model may use the pretrained feature extractor of the pretrained PPG blood pressure estimation model and may be a model that is generated by newly training the classifier using rPPG signals. The rPPG blood pressure estimation model may use a part of the pretrained feature extractor of the pretrained PPG blood pressure estimation model and may be a model that is generated by newly training a part of the feature extractor and the classifier using rPPG signals. The rPPG blood pressure estimation model may be a model that is generated by newly training the feature extractor and the classifier using rPPG signals.

The processor 140 may estimate diastolic blood pressure and systolic blood pressure using the rPPG blood pressure estimation model.

A systolic phase (SP) may be the interval from the start time of a cycle to the time of the maximum blood pressure value in the cycle. A diastolic phase (DP) may be the interval from the time of the maximum blood pressure value in a cycle to the time of the end of the cycle.

Meanwhile, the device 100 for estimating blood pressure in a non-contact manner may be provided in a portable form with the imaging unit 120, for example, one form of a mobile smartphone and a tablet device.

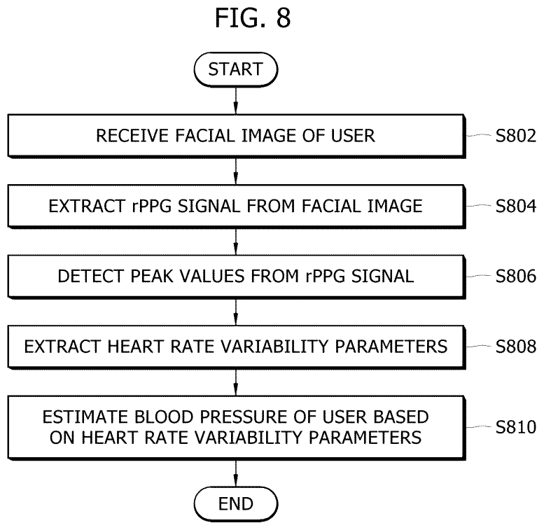

FIG. 8 is a flowchart illustrating a method of estimating blood pressure in a non-contact manner according to an exemplary embodiment of the present invention.

Referring to FIG. 8, when a facial image of a user is received from the imaging unit 120 (S802), an rPPG signal is extracted from the facial image (S804). In this case, the processor 140 may extract an rPPG signal after refining brightness variations of a skin region in the facial image.

When operation S804 is performed, the processor 140 detects a plurality of peak values from the rPPG signal (S806). For example, the processor 140 may detect R-peak values using a QRS wave detection algorithm. The processor 140 may detect a plurality of R waves during a measurement time using the QRS wave detection algorithm and sequentially detect R-peak values which are peak values of the R waves. Also, the processor 140 may detect R-peak values using the Pan & Tompkins algorithm or the Hamilton & Tompkins algorithm.

When operation S806 is performed, the processor 140 extracts HRV parameters using the detected peak values (S808). In this case, the processor 140 may extract HRV parameters of the time domain and HRV parameters of the frequency domain using the peak values of the rPPG signal. The HRV parameters of the time domain may include SDNN, SDANN, SD, RMSSD, pNN50, etc. The HRV parameters of the frequency domain may include TP, VLF, LF, HF, an LF-to-HF ratio, etc.

When operation S808 is performed, the processor 140 may estimate blood pressure of the user on the basis of the HRV parameters (S810). Here, the processor 140 may classify the blood pressure of the user as at least one of low blood pressure, normal blood pressure, and high blood pressure on the basis of HRV parameters of a specific time. Also, the processor 140 may estimate diastolic blood pressure and systolic blood pressure using time-series variation data of HRV parameters.

Specifically, the processor may input values of the HRV parameters of the specific time or log values of the HRV parameters of the specific time to the deep learning model to classify the blood pressure of the user as at least one of low blood pressure, normal blood pressure, and high blood pressure.

In addition, the processor 140 may input the time-series variation data of HRV parameters to a time-series deep learning model to estimate diastolic blood pressure and systolic blood pressure. The time-series deep learning model may be a model generated through transfer learning of a pretrained PPG blood pressure estimation model. For example, the time-series deep learning model may use a pretrained feature extractor of the pretrained PPG blood pressure estimation model and may be a model that is generated by newly training a classifier using rPPG signals. The time-series deep learning model uses a part of the pretrained feature extractor of the pretrained PPG blood pressure estimation model and may be a model that is generated by newly training a part of the feature extractor and the classifier using rPPG signals. The time-series deep learning model may be a model that is generated by newly training the feature extractor and the classifier using rPPG signals.

With a device and method for estimating blood pressure in a non-contact manner according to some exemplary embodiments of the present invention, it is possible to extract an rPPG signal from a facial image captured through an imaging module (e.g., a camera) and estimate blood pressure in a non-contact manner on the basis of the rPPG signal.

As used herein, the term “unit” may include a unit implemented in hardware, software, or firmware, and may be used interchangeably with, for example, logic, a logic block, a part, a circuit, or the like. A “unit” may be a minimum unit of an integrated part or a part thereof or may be a minimum unit for performing one or more functions or a part thereof. For example, according to an embodiment, a “unit” may be implemented in the form of an ASIC.

Although the present invention has been described above with reference to embodiments illustrated in the drawings, the embodiments are merely illustrative, and those skilled in the art should understand that various modifications and other equivalent embodiments can be made from the embodiments. Therefore, the technical scope of the present invention should be determined from the following claims.

What is claimed is:

1. A device for estimating blood pressure in a non-contact manner, the device comprising:
a memory;
an imaging unit configured to capture a body part image of a user; and
a processor connected to the memory and the imaging unit,
wherein the processor extracts a remote photoplethysmography (rPPG) signal from the body part image, detects a plurality of peak values from the rPPG signal, extracts heart rate variability (HRV) parameters using the detected peak values, and estimates blood pressure of the user on the basis of the HRV parameters.

2. The device of claim 1, wherein the body part image includes a facial image of the user.

3. The device of claim 1, wherein the processor extracts HRV parameters of a time domain and HRV parameters of a frequency domain using the peak values of the rPPG signal.

4. The device of claim 1, wherein the processor inputs the HRV parameters to a deep learning model to classify the blood pressure of the user as at least one of low blood pressure, normal blood pressure, and high blood pressure.

5. The device of claim 4, wherein the processor inputs value of the HRV parameters of a specific time or log values of the HRV parameters of the specific time to the deep learning model.

6. The device of claim 1, wherein the processor inputs time-series variation data of the HRV parameters to a time-series deep learning model to estimate diastolic blood pressure and systolic blood pressure.

7. The device of claim 6, wherein the time-series deep learning model is a model generated through transfer learning of a pretrained PPG blood pressure estimation model.

8. The device of claim 7, wherein the time-series deep learning model uses a pretrained feature extractor of the pretrained PPG blood pressure estimation model and a model generated by newly training a classifier using rPPG signals.

9. The device of claim 7, wherein the time-series deep learning model uses a part of a pretrained feature extractor of the pretrained PPG blood pressure estimation model and is a model generated by newly training a part of the feature extractor and a classifier using rPPG signals.

10. The device of claim 7, wherein the time-series deep learning model is a model generated by newly training a feature extractor and a classifier using rPPG signals.

11. The device of claim 1, further comprising a user interface unit,
wherein the processor outputs information about the estimated blood pressure through the user interface unit.

12. A method of estimating blood pressure in a non-contact manner, the method comprising:
extracting, by a processor, a remote photoplethysmography (rPPG) signal from a body part image of a user acquired through an imaging unit;
detecting, by the processor, a plurality of peak values from the rPPG signal;
extracting, by the processor, heart rate variability (HRV) parameters using the detected peak values; and
estimating, by the processor, blood pressure of the user on the basis of the HRV parameters.

13. The method of claim 12, wherein the body part image includes a facial image of the user.

14. The method of claim 12, wherein the extracting of the HRV parameters comprises extracting, by the processor, HRV parameters of a time domain and HRV parameters of a frequency domain using peak values of the rPPG signal.

15. The method of claim 12, wherein the estimating of the blood pressure of the user comprises inputting, by the processor, the HRV parameters to a deep learning model to classify the blood pressure of the user as at least one of low blood pressure, normal blood pressure, and high blood pressure.

16. The method of claim 15, wherein the estimating of the blood pressure of the user comprises inputting, by the processor, values of the HRV parameters of a specific time or log values of the HRV parameters of the specific time to the deep learning model.

17. The method of claim 12, wherein the estimating of the blood pressure of the user comprises inputting, by the processor, time-series variation data of the HRV parameters to a time-series deep learning model to estimate diastolic blood pressure and systolic blood pressure.

18. The method of claim 17, wherein the time-series deep learning model is a model generated through transfer learning of a pretrained PPG blood pressure estimation model.

19. The method of claim 18, wherein the time-series deep learning model uses a pretrained feature extractor of the pretrained PPG blood pressure estimation model and is a model generated by newly training a classifier using rPPG signals.

20. The method of claim 18, wherein the time-series deep learning model uses a part of a pretrained feature extractor of the pretrained PPG blood pressure estimation model and is a model generated by newly training a part of the feature extractor and a classifier using rPPG signals.
